(19) United States
(12) Patent Application Publication
(10) Pub. No.: US 2016/0100773 A1
(43) Pub. Date: Apr. 14, 2016

(54) PATIENT-SPECIFIC GUIDES TO IMPROVE POINT REGISTRATION ACCURACY IN SURGICAL NAVIGATION

(71) Applicant: UNIVERSITY OF WASHINGTON THROUGH ITS CENTER FOR COMMERCIALIZATION, Seattle, WA (US)

(72) Inventors: Randal P. CHING, Seattle, WA (US); Jedediah K. WHITE, Seattle, WA (US); Ernest U. CONRAD, Seattle, WA (US)

(21) Appl. No.: 14/785,836

(22) PCT Filed: Apr. 22, 2014
(86) PCT No.: PCT/US2014/034901

§ 371 Date: Oct. 20, 2015

Related U.S. Application Data
(60) Provisional application No. 61/814,604, filed on Apr. 22, 2013.

(51) Int. Cl. A61B 5/06

(52) U.S. Cl. CPC A61B 5/066; A61B 2019/527

(57) ABSTRACT

The present technology relates generally to devices for electronically registering a surgical patient to his or her own pre-surgical image scan and associated systems and methods. In some embodiments, a surgical navigation registration device configured in accordance with the technology comprises a patient-specific inner surface having at least one contour element that matches a contour of an outer surface of at least a portion of the patient's anatomy, and at least three registration pathways incorporated in the device and configured to mate with the tip of a surgical navigation system tool, such as a pointer. The registration pathways may be configured to enable the tip of the surgical navigation system tool to directly contact the patient's anatomy or, alternatively, to prevent the tip of the surgical navigation system tool from directly contacting the patient's anatomy. The registration pathways may comprise channels that extend through the patient-specific inner surface.

### CROSS-REFERENCE TO RELATED APPLICATION

This application claims priority to pending U.S. Provisional Application No. 61/814,604, filed Apr. 22, 2013, which is incorporated herein by reference in its entirety.

### TECHNICAL FIELD

The present technology relates generally to surgical navigation registration devices and methods of using surgical navigation registration devices to register a patient to his or her own pre-surgical image scan.

### BACKGROUND

Surgical navigation, also referred to as computer-assisted surgery, improves the integration of preoperative planning with intraoperative execution, thus reducing errors and variability in surgical procedures. To enable surgical navigation to correctly work, a patient's physical location is determined in three-dimensional space and aligned with a preloaded anatomical “map” based on the patient's own preoperative imaging scans. However, alignment (referred to as “registration”) of the patient frequently introduces error into the process, often requiring additional procedures (e.g., another surgery or more scans) to avoid or correct these errors. Improved devices and methods of accurately registering a surgical patient are needed.

### BRIEF DESCRIPTION OF THE DRAWINGS

Many aspects of the present technology can be better understood with reference to the following drawings. The relative dimensions in the drawings may be to scale with respect to some embodiments. With respect to other embodiments, the drawings may not be to scale. For ease of reference, throughout this disclosure identical reference numbers may be used to identify identical or at least generally similar or analogous components or features.

FIG. 1A is a perspective view of a surgical navigation registration device configured in accordance with an embodiment of the present technology before or after mating the device with a patient's bony tissue.

FIG. 1B is a perspective view of the inner (e.g., contoured) surface of the of the surgical navigation registration device of FIG. 1A.

FIG. 1C is a perspective view of the surgical navigation registration device of FIGS. 1A and 1B mated with an outer surface of a patient's bone in accordance with an embodiment of the present technology

FIG. 1D is a cross-sectional view of a portion of the surgical navigation registration device shown in FIG. 1C.

FIG. 1E is a perspective view of a surgical navigation registration device configured in accordance with another embodiment of the present technology mated with an outer surface of a patient's bone.

FIG. 2 is a perspective view of a second surgical navigation registration device mated with an outer surface of a bone in accordance with another embodiment of the present technology.

FIGS. 3A-3D are side cross-sectional views of portions of surgical navigation registration devices configured in accordance with embodiments of the present technology.

FIG. 4A is a perspective view of a surgical navigation system configured in accordance with an embodiment of the present technology.

FIG. 4B is an expanded perspective view of a portion of the surgical navigation system shown in FIG. 4A.

FIG. 5 is a flow diagram illustrating a method of electronically registering a patient for a computer-assisted surgery in accordance with an embodiment of the present technology.

### DETAILED DESCRIPTION

The present technology is generally directed to surgical navigation registration devices and methods of using such devices. Surgical navigation registration devices configured in accordance with embodiments of the present technology are expected to enhance the efficacy and/or reduce the costs associated with computer-assisted surgeries. Further, devices and methods configured in accordance with the present technology are expected to provide more reliable and/or practical surgical processes and/or surgical patient experiences relative to conventional screening devices and methods.

The current “gold standard” for electronically registering a patient includes the use of fiducial markers that are temporarily implanted in the patient's anatomy in a procedure separate from and prior to the therapeutic surgical procedure. The extra surgery required to implant the fiducial markers, however, increases the patient's risk of developing complications including, for example, infections, and also increases operating room time and overall cost. Alternatively, other techniques such as anatomical landmark registration and surface (contour) matching have been used. However, these techniques are much less accurate than fiducial markers. Intraoperative imaging is another commonly-used technique, but increases the patient's exposure to radiation and requires an operating room equipped with an intraoperative scanner.

Accordingly, the present technology is generally directed to devices, systems, and methods for electronically aligning (“registering”) a surgical patient to his or her own pre-operative image scan(s), optionally without the use of fiducial markers or intraoperative scans using imaging systems. In one embodiment, for example, a surgical navigation registration device includes a patient-specific inner surface having a contour that matches a contour of an outer surface of at least a portion of a patient's anatomy, such as a bone, and at least three registration pathways incorporated in the device and configured to mate with the tip of a surgical navigation system tool, such as a pointer. The registration pathways can include a channel extending through the device and may be optionally configured to allow the tip of the surgical navigation tool to directly contact the patient's anatomy. Alternatively, the registration pathways can protrude from a second surface of the sheet opposite the patient-specific inner surface, and may optionally be configured to prevent the tip of the surgical navigation system tool from directly contacting the patient's anatomy.

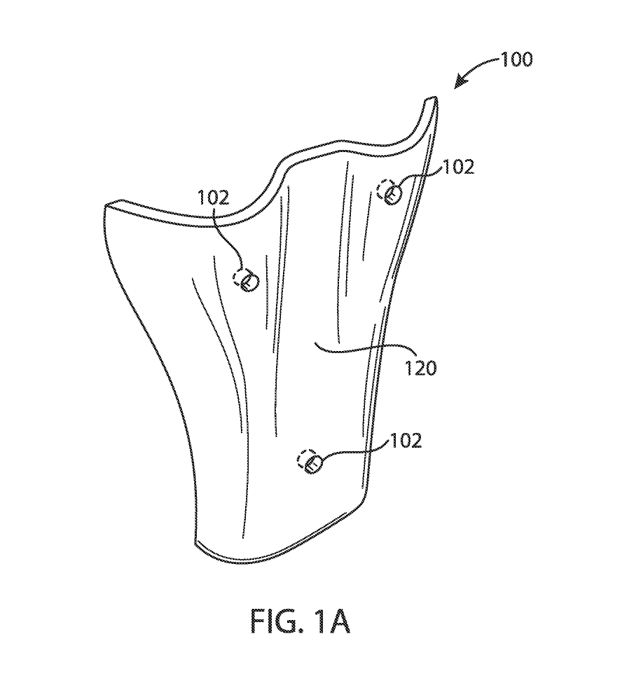

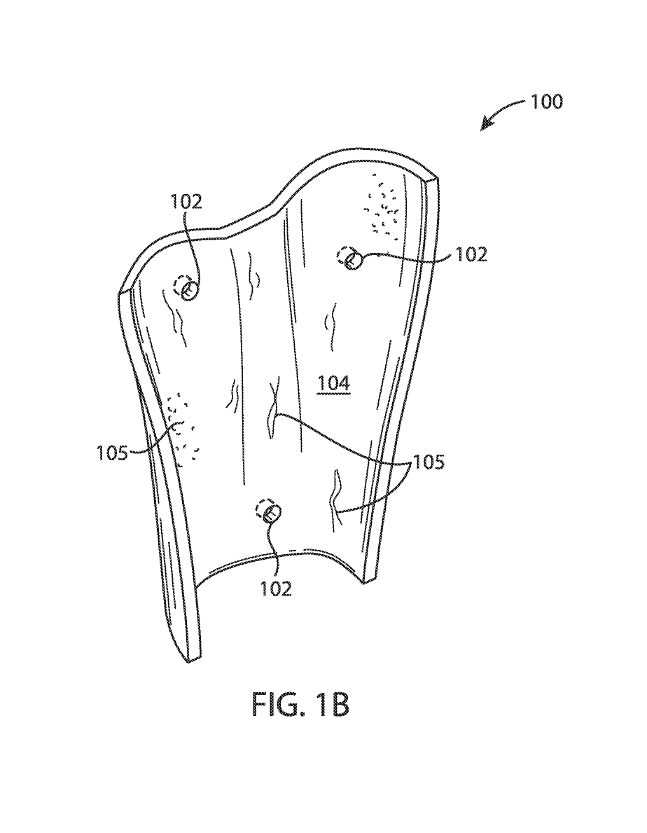

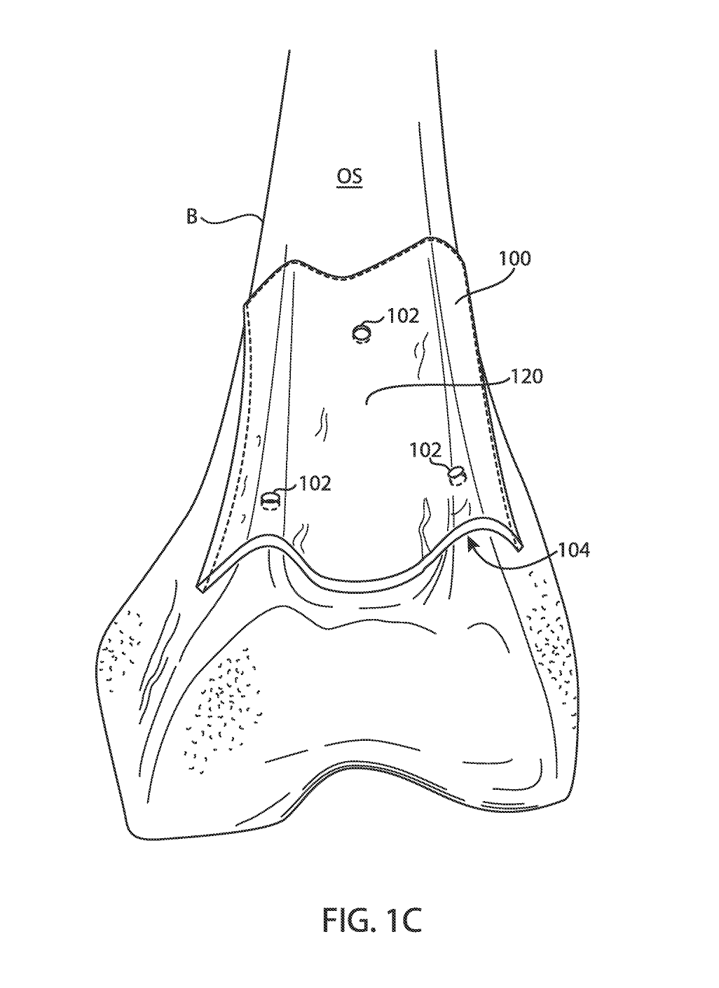

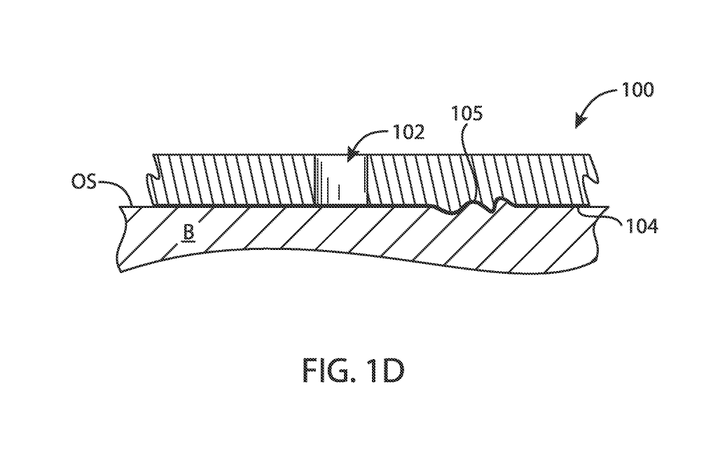

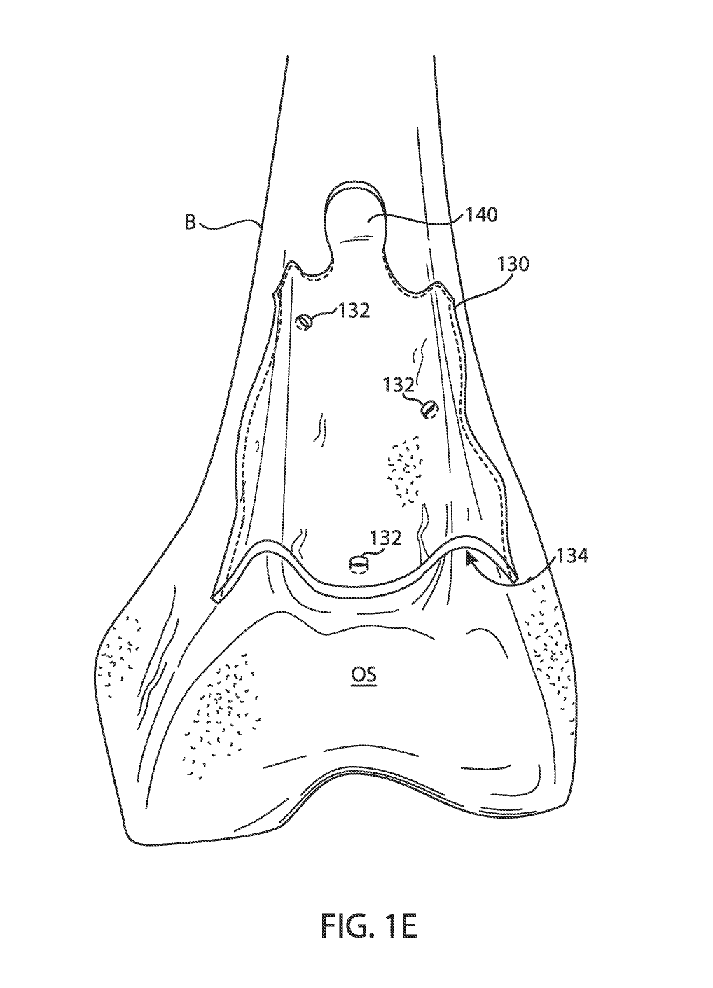

Surgical navigation registration devices consistent with the present technology, for example, may be configured to match a portion of a patient's bony tissue, cartilage, soft tissue (e.g., tendons, ligaments, etc.), organs, or a combination thereof. The portion of the patient's anatomy may include, or may be associated with, a surgical target such as a lesion, a tumor (e.g., a sarcoma), a cyst, an injury, an abscess, or any other indication requiring site-specific surgery. In some embodiments, the surgical target is covered or substantially covered by the surgical navigation registration device when in use. In other embodiments, the surgical target is located adjacent to or near the surgical navigation registration device when in use. Specific details of several embodiments of the present technology are described herein with reference to FIGS. 1-6. Although many of the embodiments are described herein with respect to surgical navigation registration guides and uses thereof, other applications and other embodiments in addition to those described herein are within the scope of the present technology. For example, some embodiments may be useful for registering patients to their pre-surgical image scans for purposes other than surgery. Moreover, a person of ordinary skill in the art will understand that embodiments of the present technology can have components and/or procedures in addition to those shown or described herein, and that these and other embodiments can be without several of the components and/or procedures shown or described herein without deviating from the present technology. The headings provided herein are for convenience only.

For ease of reference, throughout this disclosure identical reference numbers are used to identify similar or analogous components or features, but the use of the same reference number does not imply that the parts should be construed to be identical. Indeed, in many examples described herein, the identically-numbered parts are distinct in structure and/or function.

Generally, unless the context indicates otherwise, the terms “distal” and “proximal” within this disclosure reference a position or direction with respect to the treating clinician or clinician's surgical tool (e.g., a surgical navigation registration tool). “Distal” or “distally” are a position distant from or in a direction away from the clinician or clinician's surgical tool. “Proximal” and “proximally” are a position near or in a direction toward the clinician or clinician's surgical tool.

### I. SELECTED EMBODIMENTS OF SURGICAL NAVIGATION REGISTRATION DEVICES

FIG. 1A is a perspective view of a surgical navigation registration device 100 (“device 100”) configured in accordance with an embodiment of the present technology before or after mating the device 100 with a patient's bony tissue. FIG. 1B is a perspective view of the device 100 showing the inverse side of that depicted in FIG. 1A, and FIG. 1C is a perspective view of the device 100 positioned on a portion of a patient's bony tissue B.

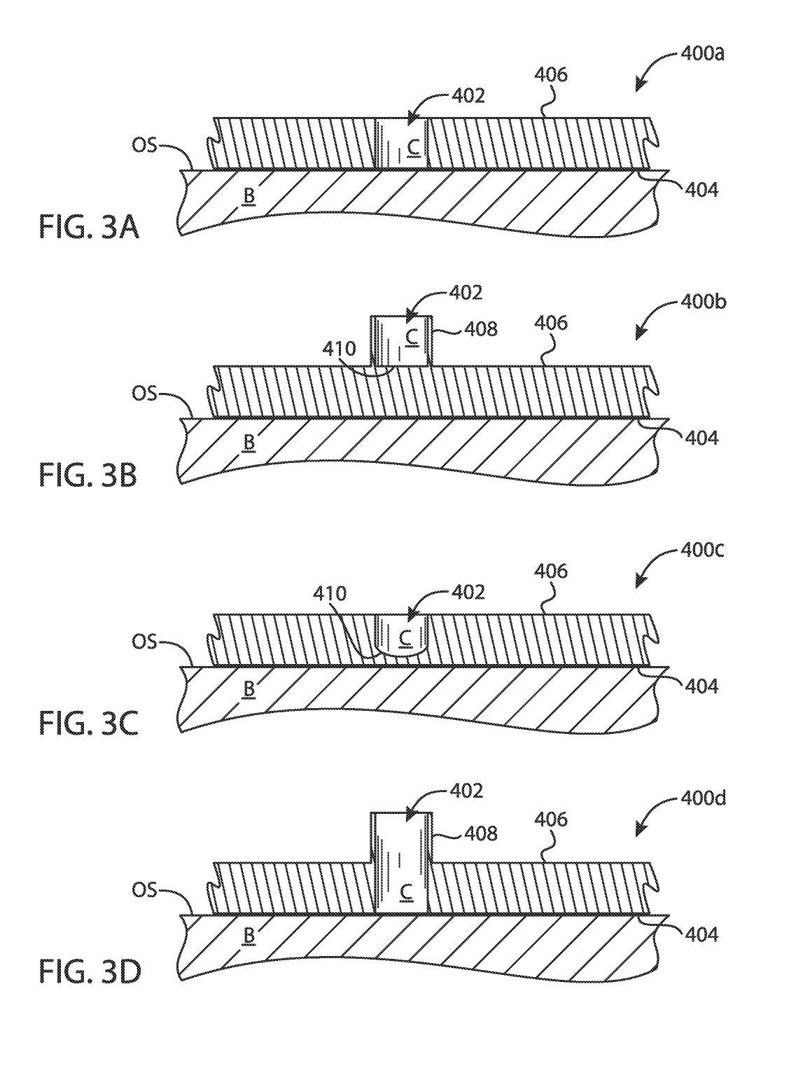

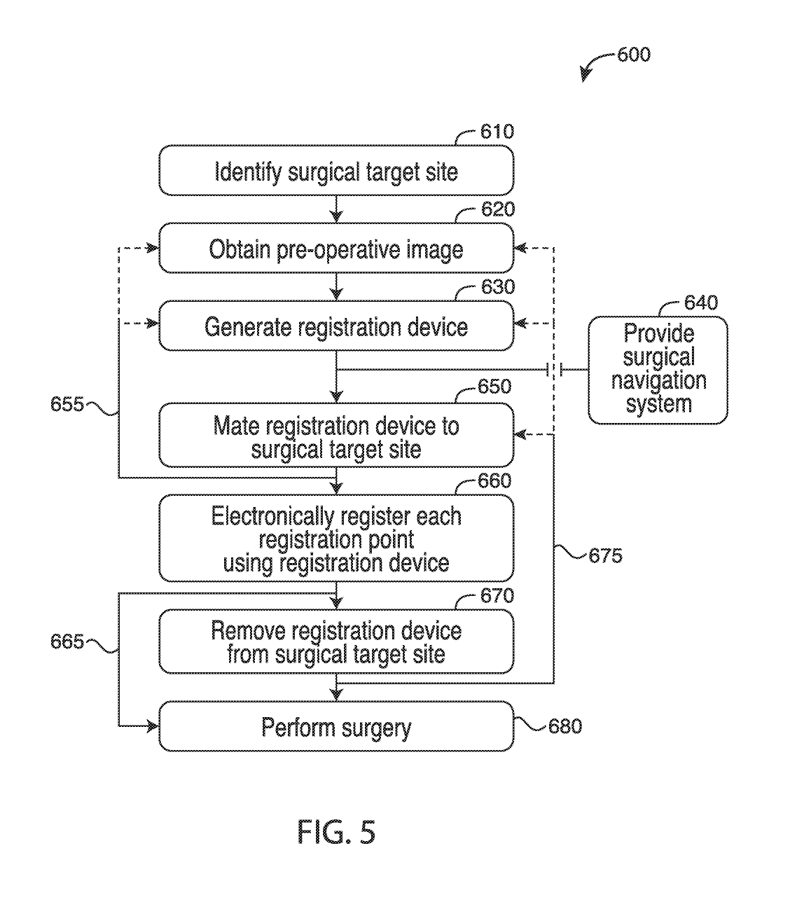

Referring to FIGS. 1A-1C together, the device 100 includes at least three registration pathways 102 and an inner surface 104. The inner surface 104 has contour elements 105 (e.g., a texture) that match, at least in part, the contour of an outer surface OS of the patient's bony anatomy B. As used herein, the term “registration pathway” refers to a feature in the device 100 configured to effectively accommodate reliable, precise, accurate and reversible insertion of a tip of a surgical navigation registration tool (e.g., a pointer) in a manner in which the position of the pointer tip effectively does not move laterally relative to the device 100. Further details regarding the use of such registration tools are provided below with reference to FIGS. 3, 5A, and 5B. The device 100 may also include a region 120 for the surgeon or clinician to grasp the device 100 or to apply pressure against the device 100 towards the bony tissue B in order to temporarily hold the device 100 in place without interfering with (e.g., blocking, hindering access to, and/or covering) any of the registration pathways 102.

The at least three registration pathways 102 may be located at any point on the device 100, although they generally must be non-collinear in arrangement. The size and shape of the registration pathways 102 may vary depending on the size and shape of a pointer tip (not shown) used to electronically register the patient. In the illustrated embodiment, for example, the registration pathways 102 are cylindrical or substantially cylindrical to accommodate a spherical pointer tip (see, e.g., element 514 in FIG. 5B). In some embodiments, a diameter or maximum inner dimension of the registration pathways 102 may be slightly larger than a diameter or maximum outer dimension of the corresponding pointer tip. In another embodiment, one or more of the registration pathways 102 may be inwardly tapered such that a diameter of an exterior opening of the registration pathway 102 at the surface distal to the bony tissue B is larger or slightly larger than a diameter of the interior opening of the corresponding registration pathway 102 at the bony tissue B. In such a configuration, the pointer tip readily enters the registration pathway 102, but can be advanced only to a pre-determined depth within the registration pathway 102. The tapered shape of the registration pathway 102 ensures that the pointer tip does not move laterally in any direction relative to the device 100 because the outermost surface of the spherical pointer tip is in complete contact with the inner wall of the registration pathway 102. In some embodiments, the predetermined depth is sufficient to allow the pointer tip to directly contact the bony tissue B; in other embodiments, however, the predetermined depth is selected to prevent the pointer tip from directly contacting the bony tissue B. In other embodiments, the registration pathways 102 may have different shapes and/or arrangements.

The size of the device 100 is large enough to include at least three registration pathways 102 separated from each other by a distance sufficient to provide meaningful electronic registration information. The minimum separation distance between registration pathways 102 depends largely on the type and sensitivity of the surgical navigation system in use. As such, the minimum separation distance will vary from device to device as will be apparent to one of skill in the art. Similarly, the device 100 is sized to readily fit the patient's anatomy without requiring a surgical incision substantially larger than would otherwise be indicated to perform the necessary surgical procedure. Because the size and location of surgical incisions varies by procedure and also from patient to patient, a person of ordinary skill in the art will readily be able to determine a maximum size of the device 100 suitable for the particular patient and surgical procedure at hand. Nonetheless, the device 100 typically has a size sufficient to provide meaningful and accurate registration thus enabling precise identification of the location of the surgical target and optionally the outer bounds of any desired surgical margins. In some embodiments, the device 100 is sized small enough to be useful as part of a minimally invasive surgery (“MIS”). In some embodiments, the device 100 may be sized sufficiently large enough to cover a portion of the patient's anatomy that has a relatively three-dimensional shape.

FIG. 1D is a cross-sectional view the device 100 positioned in contact with the outer surface OS of the patient's bony tissue B. As best seen in FIG. 1D, the contour elements 105 on the inner surface 104 match contour elements of the outer surface OS of the patient's anatomy B. The contour elements 105 may be determined from any suitable source information including, for example, a casting, a sketch, a photograph, or a pre-operative image scan (e.g., a CT scan, an MRI image, an x-ray, or a combination thereof). In one embodiment, for example, the contour elements 105 are determined from a non-invasive source, such as a pre-operative image scan. In still other embodiments, other suitable techniques may be used to determine the contour elements 105.

The device 100 is shaped and sized to be removably mated with the patient's bony tissue B, yet sufficiently stable in its association with the patient's bony tissue B that no anchors or adhesives are required to effectively secure the device 100 for use during a registration process. The shape/size of the device 100 may be determined based on a number of factors including, for example, the location of the surgical target, the type of surgical target, the size of the surgical target, the type and location of the patient's anatomical tissues in proximity to the surgical target, the likely surgical route required to reach the surgical target, the surgical tools available and/or required to perform the surgery, and other relevant factors that would be apparent to one of ordinary skill in the art. Generally, however, the device 100 has a shape/size sufficient to provide a unique removably-mated orientation with respect to the bony tissue B. As shown in FIG. 1C, for example, the device 100 has a three-dimensional shape that significantly wraps around a portion of the three-dimensional structure of the bony tissue B. In other embodiments, however, the device 100 may be configured to be removably anchored to the patient's bony tissue B with adhesive and/or temporary anchor(s).

FIG. 1E is a perspective view of a device 130 configured in accordance with another embodiment of the present technology and positioned on a portion of the patient's bony tissue B. The device 130 may be generally similar to the device 100 described above. For example, the device 130 includes at least three registration pathways 132 and an inner surface 134 having contour(s) that matches contour(s) of the outer surface OS of at least a portion of the patient's anatomy. The device 130 differs from the device 100 in that the device 130 includes a tab 130 for the surgeon or clinician to grasp the device 130 or to apply pressure against the device 130 towards the bony tissue B in order to temporarily hold the device 130 in place without interfering with (e.g., blocking, hindering access to, and/or covering) any of the registration pathways 132.

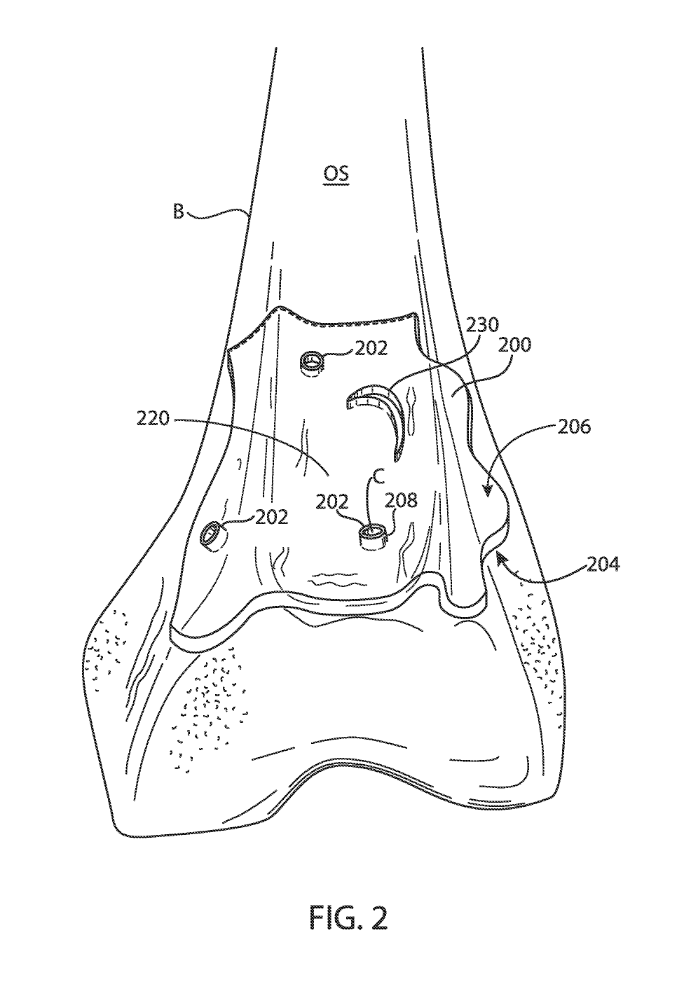

FIG. 2 is a perspective view of another surgical navigation registration device 200 (“device 200”) configured in accordance with an embodiment of the present technology and positioned on a portion of a patient's bony tissue B. The device 200 is sized and shaped to mate with at least a portion of the patient's bony anatomy B, and includes at least three registration pathways 202, an inner surface 204 matching the outer surface OS of the patient's bony anatomy B, and an outer surface 206 opposite the inner surface 204. In this embodiment, the registration pathways 202 protrude outwardly away from an external surface 206 of the device 200. In still other embodiments, however, the registration pathways 202 may have other sizes, shapes and/or profiles to accommodate pointer tips of other sizes and/or shapes. For example, the at least three registration pathways 202 may protrude outwardly away from an outer surface 206 of the device 200 via one or more plateaus, wherein each plateau includes more than one registration pathway 202.

The device 200 further includes one or more boundary features 230 configured to indicate a desired or an undesired anatomical region. For example, the boundary feature 230 may indicate to the surgeon the edge of a tumor or an expected tumor margin. Alternatively, a boundary feature 230 may indicate a region of the patient's anatomy to avoid, for example an artery, a vein, or a nerve. In some embodiments, the boundary feature 230 may indicate a region in which the patient's anatomy includes hardware incompatible with a surgical tool and/or the planned surgical procedure. The boundary features 230 may be included at any suitable location of device 200, and may be any suitable shape or configuration to effectively communicate information to the surgeon about the adjacent anatomy. The boundary feature 230 shown in FIG. 2, for example, includes a slot which extends through inner surface 204 and outer surface 206; however, the boundary feature 230 may have a variety of other suitable shapes, sizes, and/or configurations. In some embodiments, the boundary feature 230 may include a texture, a symbol, text, or other indicia describing the purpose of the boundary feature 230, and may be included on inner surface 204 and/or outer surface 206. In some embodiments, at least a portion of one or more edges of the device 200 has a shape defining, at least in part, the boundary feature 230. A boundary feature similar to boundary feature 230 may be incorporated into any device disclosed herein (e.g., device 100 and/or device 130).

The devices 100/130/200 described above with reference to FIGS. 1A-2 may be formed from a variety of suitable materials. In some embodiments, for example, the devices 100 or 200 may comprise, consist essentially of, or consist of a sterilizable material, such as a sterilizable ABS plastic and/or an acrylic material (e.g., a transparent polymer material sold under the trademark VEROCLEAR). In some embodiments, the device 100 or 200 may comprise, consist essentially of, or consist of a biocompatible and sterilizable material, for example when the device 100 or 200 is to be placed inside the patient's body during registration. A variety of other sterilizable and biocompatible sterilizable materials may also be employed; however, a person of skill in the art will recognize that such materials should be sufficiently rigid, yet flexible enough to resist deforming and fracturing during use.

FIGS. 3A-3D are side cross-sectional views of surgical navigation registration devices 400a-d configured in accordance with various embodiments of the present technology. The devices 400a-d each include registration pathways 402 and an inner surface 404 positioned to contact an outer surface OS of a patient's bony tissue B. The individual registration pathways 402 comprise a channel C, a projection 408 extending from a top surface 406, an indentation D, and/or an inner contour 410.

Referring first to FIG. 3A, the registration pathway 402 of the device 400a comprises a channel C extending from the top surface 406 through the inner surface 404. In the embodiment shown in FIG. 3B, the registration pathway 402 of the device 400b comprises a projection 408 extending outwardly away from the top surface 406 to define a channel C that extends through the projection 408, but not completely through the device 400b to the inner surface 404. Alternatively, as shown in FIG. 3C, the registration pathway 402 of the device 400c comprises a channel C extending from the top surface 406 to an intermediate depth within the device 400, but does not extend to the inner surface 404. In the embodiment shown in FIG. 3D, the registration pathway 402 of the device 400d comprises a projection 408 extending outwardly away from the top surface 406 to define a channel C that extends through the projection 408 and completely through the device to the inner surface 404.

In embodiments in which the channel C does not extend to the inner surface 404 (as shown in FIGS. 3B and 3C), the channel C may include a bottom surface 410. The bottom surface 410 may have any suitable shape. In the embodiment shown in FIG. 3B, for example, bottom surface 410 is substantially flat and substantially parallel with the inner surface 404 and/or the top surface 406. In embodiments where the shape of the device 400 precludes a substantially flat inner surface 404, the bottom surface 410 may be substantially tangential to the curvature of the portion of the inner surface 404 directly below the registration pathway 402. Referring now to FIG. 3C, the bottom surface 410 may be curved. For example, the bottom surface 410 may be concave relative to the portion of the inner surface 404 directly below the registration pathway 402. Alternatively, the bottom surface 410 may be convex, slanted, skewed, conical, or otherwise substantially not parallel to the portion of the inner surface 404 directly below the registration pathway 402. In still other embodiments, registration pathways 402 have other sizes, shapes and/or profiles to effectively accommodate reliable, precise, accurate and reversible insertion of a pointer tip.

### II. SELECTED EMBODIMENTS OF SURGICAL NAVIGATION SYSTEMS

Surgical navigation registration devices as described herein (e.g., device 100, 130, 200, 400a-d) may be incorporated into surgical navigation systems for electronically registering at least a portion of a patient's anatomy (e.g., a bony tissue surface) for a surgical procedure. Accordingly, systems consistent with the present technology include a surgical navigation registration device, a surgical navigation tool, and a processor configured to track the location of the surgical navigation tool with respect to the patient's anatomy.

Systems disclosed herein may use a computer having tracking hardware, tracking software, and a tracking device in communication with the computer and configured to receive signals from the surgical navigation tool. The systems may include, for example, one or more files including data identifying the location of one or more registration sites on the surgical navigation registration device relative to the patient's bone. The tracking device may be located at any suitable location relative to the patient, the surgical navigation registration devices as described herein, and/or the surgical navigation tool in order to effectively track the location of the tool relative to the patient's anatomy. The tracking device may be mounted to a housing, to a component of the system (e.g., a cabinet, a delivery cart, a frame, one or more articulating arms, etc.) and/or to any other suitable structure for stabilizing and/or isolating the location of the tracking device relative to the patient's anatomy.

Although not required, aspects of the technology may be described in the general context of computer-executable instructions, such as routines executed by a general-purpose computer (e.g., surgical navigation systems). Aspects of the technology can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network. In a distributed computing environment, program modules may be located in both local and remote memory storage devices.

Computer-implemented instructions, data structures, screen displays, and other data under aspects of the technology may be stored or distributed on computer-readable storage media, including magnetically or optically readable computer disks, as microcode on semiconductor memory, nanotechnology memory, organic or optical memory, or other portable and/or non-transitory data storage media. In other embodiments, aspects of the technology may be distributed over the Internet or over other networks (including wireless networks), on a propagated signal on a propagation medium (e.g., an electromagnetic wave(s), a sound wave, etc.) over a period of time, or may be provided on any analog or digital network (packet switched, circuit switched, or other scheme).

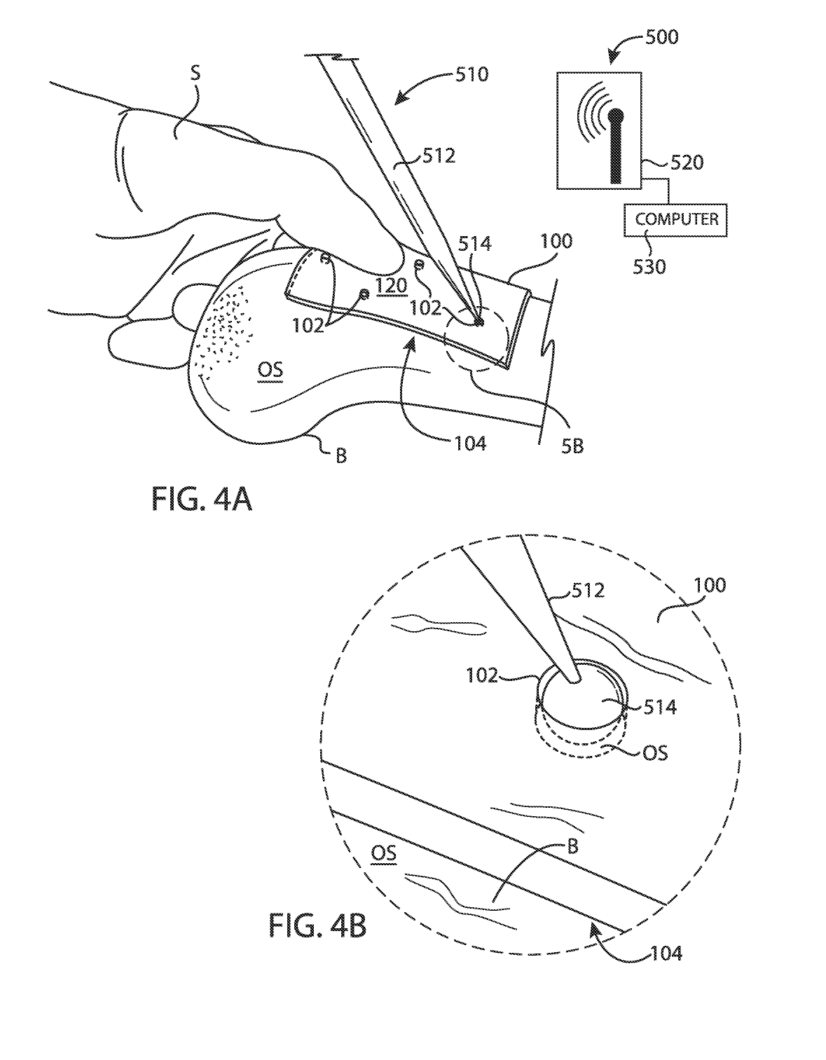

FIGS. 4A and 4B, for example, illustrate a surgical navigation system 500 configured in accordance with an embodiment of the present technology. The surgical navigation system 500 includes the surgical navigation registration device 100, a surgical navigation tool 510, and a tracking device 520. The surgical navigation registration device 100 is configured to be mated to a portion of the patient's anatomy (e.g., bony tissue B), for example by being held in place by a surgeon S. The surgical navigation registration device 100 is configured to orient the surgical navigation tool 510 in a predetermined location relative to the patient's anatomy (e.g., bony tissue B).

The tracking device 520 is in communication with a computer 530 and is configured to calibrate the location of a surgical tool relative to the patient's anatomy B based on information obtained during registration of the patient's anatomy using a surgical navigation tool 510 and a device 100 configured in accordance with the present technology. The tracking device 520 may be any suitable tracking device including, for example, commercially available tracking devices sold under the trade name UNIVERSAL TRACKER (Stryker Corporation, Kalamazoo, Mich.).

The tracking device 520 and surgical navigation tool 510 may be connected or communicatively coupled to the computer 530 via a suitable link (e.g., a wired connection, a wireless connection, a local area network (LAN), an Internet-based connection, and/or other suitable transmission means). The computer may include instructions (e.g., stored in a memory device) for interpreting data transmitted by the tracking device 520 and converting the data into information describing the position of the surgical navigation tool 510 and/or a surgical tool relative to the patient's anatomy.

The surgical navigation tool 510 may be any suitable surgical navigation tool capable of identifying a point on the patient's anatomy and operating with a surgical navigation system to match the point to a pre-operative image scan associated with the patient's anatomy. The surgical navigation tool 510 may include a pointer 512 having a tip 514. As shown in FIG. 4B, the tip 514 may be configured to contact a portion of the patient's anatomy, such as an outer surface OS of a bony tissue B. In some embodiments, the surgical navigation tool 510 is a commercially available surgical navigation tool, such as that sold under the trade name ORTHOGRIP POINTER (Stryker Corporation, Kalamazoo, Mich.).

Surgical navigation system 500 may include additional components, such as a monitor, a patient tracker, a camera, mounting hardware, a power supply, a 3D printer, and the like. Any component, or any configuration or combination of components, may be modified to suit the particular requirements of the planned surgery without deviating from the scope of the present technology.

### III. SELECTED METHODS FOR ELECTRONICALLY REGISTERING A SURGICAL PATIENT

FIG. 5 is a flow diagram illustrating a method 500 of electronically registering a patient for a computer-assisted surgery in accordance with an embodiment of the present technology. The method 500 may be used with the surgical navigation registration devices 100/130/200/400a-d described herein, or other suitable surgical navigation registration devices configured in accordance with aspects of the technology.

The method 500 of electronically registering a surgical patient begins at step 610 by identifying a surgical target site. The identification step 610 may include, for example, any suitable diagnostic test, procedure, scan, examination, or other assessment of the surgical candidate to determine at least a portion of the patient's anatomy requiring a surgical procedure.

The method 600 continues at step 620 by obtaining a pre-operative image. The pre-operative image may include, for example, a CT scan, an MRI image, an x-ray image, a bone scan, a PET scan, any other suitable image, or a combination thereof. The pre-operative image may include information about the patient's anatomy including, for example, a shape and size of the patient's anatomy adjacent to and/or in proximity to the surgical target site. The pre-operative image may further include information about one or more contour elements (e.g., contour elements 105 as shown in FIGS. 1A-1D) on the outer surface of the patient's anatomy. Alternatively, obtaining the pre-operative image as recited in step 620 may occur before the identification step 610 or as a component of the process of identifying the surgical target site (step 610). Step 620 may also be performed with the patient's anatomy positioned in a mold or casting to orient the anatomy in a certain orientation that can later be reproduced during registration.

The pre-operative image obtained in step 620 may then be used to generate a surgical navigation registration device (e.g., devices 100 or 200) in step 630. In some embodiments, the pre-operative image is processed using software (e.g., image segmentation software) to create a three-dimensional anatomical model (e.g., a virtual anatomical model). The model may then be used to create a computer-aided design (CAD) model of the surgical navigation registration device (e.g., device 100 or 200) using CAD software. For example, the CAD model may be developed using a Boolean subtraction technique based on pre-operative CT scans on the patient's anatomy. The clinician may use the CAD software to select the shape and size of the CAD model, and may locate at least three non-collinear registration pathways (e.g., registration pathways 102 and/or 202) on the device. Alternatively, the three or more non-collinear registration pathways may be randomly located on the device, for example by a randomizing component of the CAD software. The at least three registration pathways are arranged such that each pathway corresponds to (e.g., guides a registration pointer tip) to a pre-planned registration point on the pre-operative image(s).

The surgical navigation registration device may then be produced from the CAD model by any suitable production means. The device may be produced by a 3D printer (e.g., a 3D printer sold under the trademark STRATASYS). Producing the device using a 3D printer is expected to provide several advantages over traditional molding processes. First, the CAD model can be directly input into the 3D printer without the need for creating a positive mold or a negative mold. In addition, 3D printer technology requires less time to produce a finished device, and also offers the clinician more opportunities to incorporate custom features into the device (e.g., region 140 shown in FIG. 1E) without requiring modification of a physical mold. Furthermore, 3D printing technology offers clinicians more flexibility in modifying the design of the device before or during a surgery to accommodate changes in the patient's anatomy that may have occurred after obtaining the pre-operative image in step 620. Thus, even after a surgical procedure has been initiated, the present technology offers the clinician the option of rapidly adjusting, recreating, redesigning and/or re-producing the surgical navigation registration device to provide a more accurate fit and/or registration, without substantially increasing patient risk from extended surgical procedure length.

Once the surgical navigation registration device has been produced in step 630, a surgical navigation system is provided in step 640. During step 640, or alternatively at any time before registration begins in step 650, the three-dimensional coordinates (e.g., x, y, z coordinates) of the registration points corresponding to each registration pathway are input into the surgical navigation system (e.g., into the surgical navigation system planning software). With the patient prepared for the surgical procedure (e.g., anesthetized), the surgeon can make the incisions required to expose the surgical target site and/or the registration site, if not coincident. In step 650, the surgical navigation registration device is then mated (e.g., applied) to the surgical target (registration) site. If the clinician is dissatisfied with the fit of the device, the device can be adjusted, recreated, redesigned and/or re-produced by repeating step 620 and/or step 630 as shown by path 655.

If the surgeon is satisfied with the fit of the device, each registration pathway of the device is electronically registered as shown in step 660. As shown in FIGS. 4A and 4B, for example, electronic registration of a registration pathway includes inserting the tip 514 of a probe 512 into the registration pathway 102. In the embodiment shown in FIGS. 4A and 4B, for example, the tip 514 is inserted into the registration pathway 102 until it directly contacts the outer surface OS of the patient's bony anatomy B. In other embodiments, however, the tip 514 may not directly contact the outer surface OS of the patient's bony anatomy B depending on the design of the registration pathway(s). Once the tip 514 of the pointer 512 is mated with the registration pathway 102, the location of the tip 514 is transmitted to the tracking device 520 and recorded and/or matched to the pre-planned point on the patient's pre-operative image by the computer 530. This process is repeated for each of the registration pathways 102 of device 100.

Referring back to FIG. 5, after all registration pathways 102 are electronically registered in step 660, the clinician may determine whether the electronic registration is sufficient to proceed with the surgical procedure (step 680). If the registration is unacceptable, steps 650 and 660 may be repeated using the same surgical navigation registration device, or a new surgical navigation registration device may be generated by repeating step 630 and/or step 620.

Although much of the disclosure in this Specification relates to surgeon-operated (manual) computer-assisted surgery, the apparatuses, methods and systems described herein may also be used for other computer-assisted surgery techniques including, for example, robotic-assisted surgery. In addition, while much of the disclosure in this Specification relates to surgical procedures on bony tissue, the apparatuses, methods and systems described herein may also be used for surgical procedures on other portions of a patient's anatomy. For example, the surgical navigation registration devices of the present technology may be used for any computer-assisted surgical procedure on any portion of a patient including craniofacial surgery, ENT surgery, joint replacement surgery, orthopedic surgery, spinal surgery, and/or neurosurgery.

### IV. EXAMPLES

### Example 1

### Determination of Error Sources in Surgical Navigation

This example determined the baseline precision of a surgical navigation system. A machined grid with known marker distances (10 mm) was used to assess performance of a commercially available surgical navigation system (ORTHOMAP 3D, Stryker Navigation System II, Stryker Corporation, Kalamazoo, Mich.) in an ideal scenario. The navigation system consisted of a system camera, a patient tracker having a dynamic reference base, and a pointer tool. A bone of interest was represented by a physical space object, in this case the machined grid. Each marker comprised an indentation that accepted the tip of the navigation pointer tool.

Three variables and their individual effects on navigation system precision were tested:

(1) The distance from the camera to the patient tracker;

(2) The distance from the patient tracker to the center of the physical space; and

(3) The number of infrared markers in use on the patient tracker and pointer.

The camera and patient tracker distances were chosen according to the minimum and maximum practical locations in a typical operating room. For the distance between camera and patient tracker, 150 cm, 200 cm, and 247 cm were chosen (150 cm being near the minimum practical distance for use in an operating room, and 247 cm being near the outer limit). Beyond 247 cm, the patient tracker and pointer were no longer visible to the camera for tool registration. The distance between the patient tracker to the center of the physical space was tested at 20 cm, 30 cm, and 40 cm (20 cm being near the closest practical distance before pointer tool and patient tracker contact and 40 cm the extreme of likely practical applications since the tracker is typically placed on the bone of interest). The final variable, number of infrared markers, was toggled between maximum and minimum possible for system registration (e.g., all lights on and half lights off).

The physical space was registered using a DICOM image set created from a 3D scan of the grid surface. Four points were selected and used for point registration with no additional surface mapping. Three-dimensional (x,y,z) coordinates were collected for all machined indentations for the 3×3 matrix and with all lights on and half lights off. This yielded 60 annotation points plus an additional five at each of the four registration pathways for a total of 80 annotation points. Using the 60 points on the grid, distances from 10 mm and 120 mm were measured. Twelve measurements from each bin were used, for a total of 144 measurements, and the absolute difference between the measured and true lengths (defined by a MicroScribe) was calculated. The RMS error for each trial was also calculated. Intra-user reliability was assessed via the six recorded annotation points (original plus five additional points), selected while maximally varying the pointer orientation.

Mean trueness and precision (mean length measurement error and confidence intervals) were calculated for each trial with the primary outcome variable RMS error. One-way ANOVA was used to evaluate for variance according to distance from camera to patient tracker and distance from patient tracker to physical space. A t-test evaluated for difference between all markers active and minimum markers active.

Results showed that precision was dependent on both distance from the camera to the patient tracker and number of infrared markers in use on the patient tracker and pointer. With all lights on, the effect of distance was minimal and precision was sub-mm for all locations; however, covering half the lights significantly decreased precision and accuracy, with errors occasionally in excess of 3 mm. These results both reveal practical suggestions for improvement of the commercial navigation system and provide us with confidence that the greatest source of error was likely due to point registration accuracy and not system precision.

### Example 2

### Accuracy of Surgical Navigation Registration Devices Compared to Anatomic Landmark Registration and Fiducial Markers

This example compared the accuracy of electronically registering a portion of a patient's anatomy using a surgical navigation registration device in accordance with the present technology to electronic registration using fiducial markers.

A “Sawbones” femur model (Pacific Research Labs, Vashon Island, Wash.) was used to compare registration accuracy of a 3D-printed point registration guide in accordance with the present technology with both anatomic landmark and fiducial marker registration. The registration guide was printed by Fathom (Seattle, Wash.) using a Stratasys 3D printer and Veroclear (acrylic) plastic. Registration accuracy was established by computing measurement error (ME) as the difference between pre-planned registration points and resampled registration points following registration for each of the three registration techniques. In addition to resampling the registration points (3 points), target accuracy was also evaluated by digitizing known target points (6 points) that were burred into the Sawbones femur prior to CT imaging. The 3D-printed point-registration guide showed comparable accuracy to fiducial-marker registration and was significantly better than anatomic landmark registration (Table 1).

TABLE 1
Registration and target accuracy for three registration techniques.

| | | |
|---|---|---|
| | Registration | Target |
| Registration | Accuracy (mm) | Accuracy (mm) |

| Technique | Mean | Std. Dev. | Mean | Std. Dev. |
|---|---|---|---|---|
| Anatomic | 3.05 | 1.89 | 5.25 | 1.07 |
| Landmark | | | | |
| Fiducial Marker | 0.52 | 0.12 | 0.76 | 0.31 |
| 3D-Printed | 0.55 | 0.39 | 0396 | 0.40 |
| Guide | | | | |

### Example 3

### Accuracy of Surgical Navigation Registration Devices Compared to Anatomic Landmark Registration and Fiducial Markers

This example compares the registration and target accuracy of the present technology with existing registration techniques in a human cadaver model.

A verification study using human cadaver lower-body specimens (pelvis-to-ankle) is performed to compare the registration and target accuracy of: (1) anatomic landmark registration (standard practice); (2) fiducial-marker registration (“gold standard”); and (3) patient-specific guides for registration in accordance with the present technology.

The verification study is performed in several anatomical locations where bone tumor resections are commonly performed including: 1) the proximal and distal femur, 2) the proximal and distal tibia, and 3) the pelvis. A minimum of five lower-body specimens are chosen to obtain data from a total of 10 lower extremities (5 bilaterally).

The study comprises the milestones/tasks shown in Table 2.

TABLE 2
Study Design.

| Milestones/Tasks | Description |
|---|---|
| Task 1: Obtain specimens | 5 lower-body cadaver specimens (pelvis-to-ankle) |
| | 10 lower extremities total (5 bilaterally) |
| Task 2: Implant fiducial | 4-mm dia. CT beads, 5 anatomic locations |
| markers (for fiducial | Pelvis |
| registration and | Femur (proximal and distal) |
| measuring target | Tibia (proximal and distal) |
| accuracy) | 9 to 12 fiducials per anatomic location |
| | 3 to 6 for fiducial---marker registration |
| | 6 for evaluation of target accuracy |
| Task 3: CT scan specimens | 512 × 512 pixel spatial matrix for each scan |
| | 0.5 × 0.5 mm in-plane dimension |
| | 0.625 mm slice thickness using bone algorithm |
| Task 4: Develop and 3D | Use image segmentation software to create computer |
| print cadaver- | model of bone from CT |
| specific navigation | Select pre-picked registration points on CT |
| guides | Generate computer-aided design (CAD) model of the |
| | point-registration guide from anatomical computer model |
| | Rapid-prototype (e.g., 3D print) the CAD model of the point- |
| | registration guide |
| Task 5: Conduct registration | Load CT study of interest into the surgical navigation |
| and target accuracy | system |
| testing | Record and evaluate registration and target accuracy for 3 |
| | different registration techniques: |
| | Anatomic landmark registration (standard practice) |
| | Fiducial marker registration (“gold standard”) |
| | 3D-printed cadaver--specific guide |
| Task 6: Analyze data | Compute measurement error (ME) and compare registration |
| and run statistical | and target accuracy for the three registration techniques. |
| analyses | |

### Example 4

### Electronic Registration of Surgical Patients

For this validation study, 3D-print patient-specific registration guides are prepared and used for 10 patients undergoing tumor resection surgery on a variety of tumor locations including, for example, including the proximal/distal femur, tibia, and humerus, and the pelvis.

To create the patient-specific navigation guides, a CAD model of each guide is created using the normal preoperative CT imaging of the patient's anatomy. The CAD models are printed on a 3D printer using an FDA-approved biocompatible, sterilizable acrylic material (e.g., MED610). Once printed, the guides are gas-sterilized and delivered to the operating room prior to surgery. The 3D coordinates of the planned registration points (corresponding to registration pathway positions on the guide) are uploaded to the surgical navigation system (e.g., a Stryker Navigation System) prior to registration.

Registration time and accuracy are recorded while using patient-specific registration guides to demonstrate that using surgical navigation registration devices configured in accordance with embodiments of the present technology significantly reduces registration time and improved registration accuracy (reduced ME) compared to conventional anatomic (landmark) registration techniques.

### V. FURTHER EXAMPLES

1. A surgical navigation registration device, comprising:
a patient-specific inner surface having a contour that matches a contour of an outer surface of at least a portion of a patient's anatomy; and
at least three registration pathways incorporated in the device, wherein each registration pathway is configured to mate with a tip of a surgical navigation system tool.

2. The surgical navigation registration device of example 1 wherein the at least three registration pathways comprise channels that extend through the patient-specific inner surface of the device.

3. The surgical navigation registration device of example 1 wherein the at least three registration pathways protrude from a second surface of the sheet opposite the patient-specific inner surface.

4. The surgical navigation registration device of example 3 wherein the at least three registration pathways are configured to prevent the tip of the surgical navigation system tool from directly contacting the patient's anatomy.

5. The surgical navigation registration device of example 1 or example 2, wherein the at least three registration pathways are configured to enable the tip of the surgical navigation system tool to directly contact the patient's anatomy.

6. The surgical navigation registration device of any one of examples 1 to 5, wherein the contour of the patient-specific inner surface is determined from a pre-operative image associated with the patient.

7. The surgical navigation registration device of any one of examples 1 to 6, wherein the contour of the patient-specific inner surface is configured such that the device fits securely to the patient in a unique orientation without use of an anchor or an adhesive.

8. The surgical navigation registration device of any one of examples 1 to 7, wherein the device is configured to allow a pre-operative or an intra-operative modification to improve fit of the device while retaining at least three registration pathways.

9. The surgical navigation registration device of example 5 wherein the tip of the surgical navigation system tool is substantially spherical or spherical and defines a diameter, and wherein the device has a thickness substantially similar to the diameter of the tip.

10. The surgical navigation registration device of any one of examples 1 to 9, wherein the guide includes at least four, at least five, or more than five registration pathways.

11. The surgical navigation registration device of example 6 wherein the pre-operative image is one or more of: MRI, CT, X-ray, bone scan, PET or other imaging modality.

12. The surgical navigation registration device of any one of examples 1 to 11, wherein the registration pathways guide the surgical navigation tool to pre-planned registration points on the outer surface of the patient's anatomy.

13. The surgical navigation registration device of example 12, wherein the pre-planned registration pathways correspond to pre-selected points in a computer-assisted navigation software model generated from a pre-operative image associated with the patient.

14. The surgical navigation registration device of any one of examples 1 to 13, wherein the registration pathways comprise a shape complimentary to a geometry of the surgical navigation system tool.

15. The surgical navigation registration device of any one of examples 1 to 14, wherein the device is be manufactured by 3D printing from a biocompatible and sterilizable material.

16. The surgical navigation registration device of any one of examples 1 to 15, wherein the device is configured to be secured during a surgical navigation registration process using a fastener and/or an adhesive.

17. The surgical navigation registration of any one of examples 1 to 16, wherein the device is configured to be repositioned for a re-registration process in a substantially same position as a previous registration process.

18. The surgical navigation registration device of any one of examples 1 to 17, wherein the device further comprises an additional registration pathway corresponding to a pre-planned location of surgical or anatomical interest.

19. The surgical navigation registration device of example 18, wherein the pre-planned location of surgical or anatomical interest comprises a tumor boundary, an osteotomy plane, or a tool trajectory.

20. The surgical navigation registration device of any one of examples 1 to 19, wherein the device further comprises a boundary feature configured to indicate a desired or an undesired anatomical region.

21. The surgical navigation registration device of example 20, wherein the desired or undesired anatomical region comprises a tumor margin.

22. A patient-specific surgical navigation registration device, comprising:
a sheet (e.g., a thin sheet or a block) configured to reversibly mate with at least a portion of a bone of a human patient; and
at least three registration pathways incorporated into the sheet,
wherein the sheet is configured to mate using a pre-operative image showing a surface of at least the portion of the bone of the patient.

23. The patient-specific surgical registration device of example 22 wherein the sheet is configured to mate with the portion of the bone without adhesive or fastener.

24. The patient-specific surgical registration device of example 22 or example 23, wherein the at least three registration pathways are channels that extend through the sheet.

25. The patient-specific surgical registration device of example 22 or example 23, wherein the at least three registration pathways protrude from a surface of the sheet opposite the bone of the patient.

26. The patient-specific surgical registration device of example 25 wherein the at least three registration pathways are configured to prevent a tip of a surgical navigation system pointer to directly contact the bone of the patient.

27. The patient-specific surgical registration device of any one of examples 22 to 25, wherein the at least three registration pathways are configured to enable a tip of a surgical navigation system pointer to directly contact the bone of the patient.

28. The patient-specific surgical registration device of example 27 wherein the tip of the surgical navigation system pointer is substantially spherical or spherical and defines a diameter, and wherein the sheet has a thickness substantially similar to the diameter of the tip.

29. The patient-specific surgical registration device of any one of examples 22 to 28, wherein the sheet includes four registration pathways.

30. The patient-specific surgical registration device of any one of examples 22 to 29, wherein the pre-operative image is one or more of the following: MRI, CT, X-ray, bone scan, PET, or other imaging modality.

31. A surgical navigation system for electronically registering at least a portion of a bony tissue surface of a subject, the system comprising:
a pointer having a registration tip, the pointer in electronic communication with a computer;
a tracking device in communication with the computer and configured to track a position of the registration tip; and
a registration guide configured to mate with at least a portion of the bony tissue surface of the subject, the registration guide comprising at least three registration pathways and a contoured inner surface derived from a pre-operative image of the subject.

32. The surgical navigation system of example 31 wherein the registration guide is configured to mate with the portion of the bony tissue surface without adhesive or fastener.

33. The surgical navigation system of example 31 or 32, wherein the at least three registration pathways are channels that extend through the registration guide.

34. The surgical navigation system of example 31 or 32, wherein the at least three registration pathways protrude from a surface of the registration guide opposite the bony tissue surface.

35. The surgical navigation system of any one of examples 31, 32 or 34, wherein the at least three registration pathways are configured to prevent the registration tip of the pointer to directly contact the bony tissue surface.

36. The surgical navigation system of any one of examples 31 to 33, wherein the at least three registration pathways are configured to enable the registration tip of the pointer to directly contact the bony tissue surface.

37. The surgical navigation system of example 36 wherein the registration tip of the pointer is substantially spherical or spherical and defines a diameter, and wherein the registration guide has a thickness substantially similar to the diameter of the registration tip.

38. The surgical navigation system of any one of examples 31 to 37, wherein the registration guide includes four, five or six registration pathways.

39. The surgical navigation system of any one of examples 31 to 38, wherein the pre-operative image is one or more of the following: MRI, CT, X-ray, bone scan, PET or other imaging modality.

40. A method of electronically registering at least a portion of a bony tissue for surgery, the method comprising:
identifying at least a portion of a bone in need of a surgical procedure;
providing or obtaining a pre-operative image including data describing a surface contour of the portion of a bony tissue surface of the bone;
selecting at least three pre-planned registration points on the pre-operative image;
generating a registration mask comprising a contoured surface and at least three registration pathways;
providing a surgical navigation system comprising a pointer including a tip;
inputting coordinates corresponding to the at least three pre-planned registration points into the surgical navigation system;
mating the registration mask with the bony tissue; and
inserting the tip into each of the at least three registration pathways to register the at least three registration points,
wherein at least a portion of the contoured surface corresponds to the surface contour of the portion of the bony tissue surface of the bone, and
wherein each of the at least three registration pathways is configured to guide the tip of the pointer to one of the pre-planned registration points.

41. The method of example 40 wherein the registration mask is configured to mate with the portion of the bone without adhesive or fastener.

42. The method of example 40 or example 41, wherein the at least three registration pathways are channels that extend through the registration mask.

43. The method of example 40 or example 41, wherein the at least three registration pathways protrude from a surface of the registration mask opposite the contoured surface.

44. The method of any one of examples 40, 41 or 43, wherein the at least three registration pathways are configured to prevent the tip of the pointer to directly contact the bony tissue surface.

45. The method of any one of examples 40 to 42, wherein the at least three registration pathways are configured to enable the tip of the pointer to directly contact the bony tissue surface.

46. The method of example 45 wherein the tip of the pointer is substantially spherical or spherical and defines a diameter, and wherein the registration mask has a thickness substantially the same as the diameter of the tip.

47. The method of any one of examples 40 to 46, wherein the registration mask includes four, five or six registration pathways.

48. The method of any one of examples 40 to 47, wherein the pre-operative image is one or more of the following: MRI, CT, X-ray, bone scan, PET or other imaging modality.

### V. CONCLUSION

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown or described in detail to avoid unnecessarily obscuring the description of the embodiments of the present technology. Although steps of methods may be presented herein in a particular order, alternative embodiments may perform the steps in a different order. Similarly, certain aspects of the present technology disclosed in the context of particular embodiments can be combined or eliminated in other embodiments. While advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the present technology. Accordingly, this disclosure and associated technology can encompass other embodiments not expressly shown or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout to mean including at least the recited feature(s) such that any greater number of the same feature and/or additional types of other features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

1. A surgical navigation registration device, comprising:
a patient-specific inner surface having a contour that matches a contour of an outer surface of at least a portion of a patient's anatomy; and
at least three registration pathways incorporated in the device, wherein each registration pathway is configured to mate with a tip of a surgical navigation system tool.

2. The surgical navigation registration device of claim 1 wherein the at least three registration pathways comprise channels that extend through the patient-specific inner surface of the device.

3. The surgical navigation registration device of claim 1 wherein the at least three registration pathways protrude from a second surface of the sheet opposite the patient-specific inner surface.

4. The surgical navigation registration device of claim 3 wherein the at least three registration pathways are configured to prevent the tip of the surgical navigation system tool from directly contacting the patient's anatomy.

5. The surgical navigation registration device of claim 1, wherein the at least three registration pathways are configured to enable the tip of the surgical navigation system tool to directly contact the patient's anatomy.

6. The surgical navigation registration device of claim 1, wherein the contour of the patient-specific inner surface is determined from a pre-operative image associated with the patient.

7. The surgical navigation registration device of claim 1, wherein the contour of the patient-specific inner surface is configured such that the device fits securely to the patient in a unique orientation without use of an anchor or an adhesive.

8. (canceled)

9. (canceled)

10. The surgical navigation registration device of claim 1, wherein the guide includes at least four, at least five, or more than five registration pathways.

11. The surgical navigation registration device of claim 6 wherein the pre-operative image is one or more of: MRI, CT, X-ray, bone scan, and PET.

12. The surgical navigation registration device of claim 1, wherein the registration pathways guide the surgical navigation tool to pre-planned registration points on the outer surface of the patient's anatomy.

13. (canceled)

14. (canceled)

15. The surgical navigation registration device of claim 1, wherein the device comprises a sterilizable and optionally biocompatible material.

16. (canceled)

17. (canceled)

18. The surgical navigation registration device of claim 1 wherein the device further comprises an additional registration pathway corresponding to a pre-planned location of surgical or anatomical interest.

19. (canceled)

20. The surgical navigation registration device of claim 1 wherein the device further comprises a boundary feature configured to indicate a desired or an undesired anatomical region.

21. (canceled)

22. A patient-specific surgical navigation registration device, comprising:
a sheet configured to reversibly mate with at least a portion of a bone of a human patient; and
at least three registration pathways incorporated into the sheet,
wherein the sheet is configured to mate using a pre-operative image showing a surface of at least the portion of the bone of the patient.

23. The patient-specific surgical registration device of claim 22 wherein the sheet is configured to mate with the portion of the bone without adhesive or fastener.

24. The patient-specific surgical registration device of claim 22, wherein the at least three registration pathways are channels that extend through the sheet.

25. (canceled)

26. (canceled)

27. The patient-specific surgical registration device of claim 22, wherein the at least three registration pathways are configured to enable a tip of a surgical navigation system pointer to directly contact the bone of the patient.

28-30. (canceled)

31. A surgical navigation system for electronically registering at least a portion of a bony tissue surface of a subject, the system comprising:
a pointer having a registration tip, the pointer in electronic communication with a computer;
a tracking device in communication with the computer and configured to track a position of the registration tip; and
a registration guide configured to mate with at least a portion of the bony tissue surface of the subject, the registration guide comprising at least three registration pathways and a contoured inner surface derived from a pre-operative image of the subject.

32. The surgical navigation system of claim 31 wherein the registration guide is configured to mate with the portion of the bony tissue surface without adhesive or fastener.

33. The surgical navigation system of claim 31, wherein the at least three registration pathways are channels that extend through the registration guide.

34. (canceled)

35. (canceled)

36. The surgical navigation system of claim 31, wherein the at least three registration pathways are configured to enable the registration tip of the pointer to directly contact the bony tissue surface.

37-48. (canceled)
